(19) United States
(12) Patent Application Publication
(10) Pub. No.: US 2022/0264813 A1
(43) Pub. Date: Aug. 25, 2022

(54) INTERSPECIFIC DIANTHUS PLANTS AND METHODS OF PRODUCING SAME

(71) Applicant: Danziger Dan Flower Farm, Moshav Mishmar HaShiva (IL)

(72) Inventors: Gregory Robert SCOVEL, Kiryat Ekron (IL); Hadas PRICE, Moshav Givat Yeshayahu (IL); Gavriel DANZIGER, Moshav Nir-Zvi (IL)

(73) Assignee: Danziger Dan Flower Farm, Moshav Mishmar HaShiva (IL)

(21) Appl. No.: 17/628,289

(22) PCT Filed: Aug. 12, 2020
(86) PCT No.: PCT/IL2020/050892

§ 371 Date: Jan. 19, 2022

Related U.S. Application Data
(60) Provisional application No. 62/885,353, filed on Aug. 12, 2019.

(51) Int. Cl. A01H 1/02; A01H 1/04; A01H 1/00; A01H 5/02; A01H 6/30

(52) U.S. Cl. CPC A01H 1/021; A01H 1/045; A01H 1/1255; A01H 5/02; A01H 6/305

(57) ABSTRACT

An interspecific Dianthus plant is provided. The interspecific plant comprises a genomic sequence distinctive of D. japonicus and a genomic sequence distinctive of Dianthus species not being D. japonicus, the plant exhibiting resistance to Fusarium oxysporum f.sp. dianthi, race 2 which is higher than that of the Dianthus species.

### RELATED APPLICATION/S

This application claims the benefit of priority of U.S. Provisional Patent Application No. 62/885,353 filed on 12 Aug. 2019, the contents of which are incorporated herein by reference in their entirety.

### SEQUENCE LISTING STATEMENT

The ASCII file, entitled 83586SequenceListing.txt, created on Aug. 11, 2020, comprising 1,690 bytes, submitted concurrently with the filing of this application is incorporated herein by reference.

### FIELD AND BACKGROUND OF THE INVENTION

The present invention, in some embodiments thereof, relates to interspecific Dianthus plants and methods of producing same.

The genus Dianthus comprises more than 300 species (Jurgens, A., et al., 2003). Although many species have been used in breeding projects over the past century, most have contributed at best, to the development of garden types (D. chinensis, D. plumarius, D. barbatus, D. deltoides). Once established as the “cultured” cut flower species in the 1930's, Dianthus caryophyllus has dominated the market ever since. Much high level breeding work has been invested in this species and today there are many, high quality varieties, available. However, despite its high popularity in the past, the Carnation (Dianthus caryophyllus) market has declined substantially in the past twenty years. Therefore a new innovative type is much sought after.

Objectives of breeding programs are defined by the problems and weaknesses of the current cultivars. Interspecific hybridization is a mean to increase genetic variability and introduce new valuable traits, thus breeding programs rely heavily on the genetic diversity of the crop.

After the definition of specific breeding objectives the next step is the selection of the parental lines that possess the traits required to meet the program goals, parents whose combination will deliver an improved cultivar.

Breeders often encounter hybridization barriers that hamper the development of interspecific hybrids, such a barrier could be the timing of flowering of the parental species. Plant species that originate from different geographic locations usually comprise different mechanisms for flowering induction, such as day length and temperature. Cultivation of these species usually requires planting in the growth conditions that characterize the natural environment in their origin. For the breeder who wishes to create an interspecific hybridization, flower timing of the parental lines can become a barrier in creating the designated cross.

Previous reports on interspecific hybridization in the genus Dianthus include mainly the species Dianthus caryophyllus L. (Carnation), some of which are described infra.

Nimura at el. 2003 have reported a successful interspecific cross between Carnation and D. japonicus. Other reports include interspecific crosses between Carnation and Dianthus deltoides, D. japonicus, Dianthus knappii or Dianthus superbus (Kanda1992), hybrids created by crosses between Carnation and Dianthus capitatus (Onozaki et al. 1998), interspecific somatic hybridization through protoplast fusion of Dianthus chinensis and D. barbatus (Nakano and Mii 1993) and so on.

D. barbatus is a well-known horticulture crop that has been around since ancient times and is native to Eurasia. This plant is commonly known as Sweet William and has a biennial life cycle. Flowers are produced in dense clusters that come in a wide range of colors, including many bicolored cultivars. Modern cultivar series may be tall for cut flower purposes or shorter for pot and bedding applications. Dianthus barbatus may suffer from Fusarium or Rhizoctonia (Greenhouse Production of Dianthus, abama Cooperative Extension System2007).

D. japonicus is a perennial species indigenous to Japan. It is described by Nimura et al. 2003, as a fasciculate cyme with many small flowers, an upright robust stem, broad and thick evergreen foliage with a developed cuticle, good heat tolerance ((Tukamoto 1968) and it does not suffer from carnation bacterial wilt (Kagito and Tuchiya 1968).

Xijun et al. Chinese Agricultural Science Bulletin, 1 Jan. 2005, 21(11):262-264 (www(dot)europepmcdotorg/article/cba/617875);

www(dot)jstage(dot)jst.godotjp/article/jsbbs/56/3/56_3_303/pdf.

### SUMMARY OF THE INVENTION

According to an aspect of some embodiments of the present invention there is provided an interspecific Dianthus plant comprising a genomic sequence distinctive of D. japonicus and a genomic sequence distinctive of Dianthus species not being D. japonicus, the plant exhibiting resistance to Fusarium oxysporum f.sp. dianthi, race 2 which is higher than that of the Dianthus species.

According to an aspect of some embodiments of the present invention there is provided an interspecific Dianthus plant comprising a genomic sequence distinctive of D. barbatus and a genomic sequence distinctive of D. japonicus.

According to some embodiments of the invention, flowers of the interspecific plant are endowed with a sweet scent and/or higher shelf life compared to that of a D. barbatus parent or about the same as that of a D. japonicus parent of the interspecific plant.

According to some embodiments of the invention, the interspecific plant exhibits resistance to Fusarium oxysporum f.sp. dianthi, race 2 which is higher than that of a D. barbatus parent or at least about the same as that of a D. japonicus parent of the interspecific plant.

According to some embodiments of the invention, the interspecific plant exhibits heat tolerance which is higher than that of a D. barbatus parent of the interspecific plant or about the same or higher than that of a D. japonicus parent.

According to some embodiments of the invention, the interspecific plant exhibits flower size which is larger than that of a D. japonicus parent of the interspecific plant.

According to some embodiments of the invention, the interspecific plant exhibits a flower size of at least 1 cm.

According to some embodiments of the invention, the interspecific plant exhibits a flower size of 1-6 cm.

According to some embodiments of the invention, the interspecific plant exhibits a stem length exceeding 60 cm.

According to some embodiments of the invention, the interspecific plant exhibits a stem length of 60-120 cm.

According to some embodiments of the invention, the interspecific plant exhibits a stem length of 15-60 cm.

According to some embodiments of the invention, the interspecific plant exhibits a single flower type.

According to some embodiments of the invention, the interspecific plant exhibits a double flower type.

According to some embodiments of the invention, the interspecific plant exhibits a semi-double flower type.

According to some embodiments of the invention, the interspecific plant exhibits a flower color selected from the group consisting of dark pink, cerise, light pink, purple and cream white.

According to some embodiments of the invention, the interspecific plant exhibits a flower pattern selected from the group consisting of plain, vein and bicolor.

According to some embodiments of the invention, the interspecific plant exhibits a flower type selected from the group consisting of a head type flower and a spray type flower.

According to some embodiments of the invention, the interspecific plant exhibits a green flower-like structure.

According to some embodiments of the invention, the interspecific plant exhibits rooting ability at least as good as that of a D. barbatus parent of the interspecific plant.

According to some embodiments of the invention, the interspecific plant is sterile.

According to some embodiments of the invention, the genomic sequence distinctive of D. barbatus is as set forth in Sequence 3 (SEQ ID NO: 3) or Sequence 4 (SEQ ID NO: 4).

According to some embodiments of the invention, the genomic sequence distinctive of D. japonicus is as set forth in Sequence 1 (SEQ ID NO: 1) or Sequence 2 (SEQ ID NO: 2).

According to an aspect of some embodiments of the present invention there is provided a cut flower of the interspecific hybrid plant as described herein.

According to an aspect of some embodiments of the present invention there is provided a cutting of the interspecific hybrid plant as described herein.

According to an aspect of some embodiments of the present invention there is provided a hybrid seed forming the interspecific hybrid plant as described herein.

According to an aspect of some embodiments of the present invention there is provided a tissue culture of the interspecific hybrid plant as described herein.

According to an aspect of some embodiments of the present invention there is provided a potted flower of the interspecific hybrid plant as described herein.

According to an aspect of some embodiments of the present invention there is provided a method of producing the interspecific Dianthus plant, the method comprising:

(a) selecting from a plurality of D. japonicus plants an early flowering D. japonicus plant;

(b) crossing the early flowering D. japonicus plant with a Dianthus species not being D. japonicus so as to obtain the interspecific Dianthus plant.

According to an aspect of some embodiments of the present invention there is provided a method of producing the interspecific Dianthus plant, the method comprising:

(a) selecting from a plurality of D. japonicus plants an early flowering D. japonicus plant;

(b) crossing the early flowering D. japonicus plant with a D. barbatus plant so as to obtain the interspecific Dianthus plant.

According to some embodiments of the invention, the method further comprises subjecting to mutagenesis the early flowering D. japonicus plants prior to the crossing.

According to some embodiments of the invention, the method further comprises selecting the interspecific plant for at least one of:

Single, semi double flower type or double flower type;

height of at least 60 cm;

height below 60 cm;

attractive and durable foliage; dark, wide, shiny leaves;

Number of stems per plant of at least 6;

heat tolerance higher than that of D. caryophyllus, and optionally than that of D japonicus; and

resistance to Fusarium oxysporum f.sp. dianthi, race 2 at least as good as that of the D. japonicus parent.

According to an aspect of some embodiments of the present invention there is provided a method of growing the interspecific Dianthus plant under conditions which allow growth of the interspecific Dianthus plant.

According to an aspect of some embodiments of the present invention there is provided a D. japonicus plant or a part thereof, representative seeds of said plant having been deposited under the Budapest Treaty at the NCIMB under NCIMB 43450 (DTJ-16-4748) on Aug. 2, 2019, progeny or hybrids of the plant.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is an image showing the rooting variation in the hybrids of some embodiments of the invention. Rooted cuttings of parental D. barbatus lines DT-Z-171, DT-Z-173 and DT-Z-174 are on the left. In the middle—Cuttings of the parental varieties of D. japonicus DTJ-16-4787 and DTJ-16-4788 colored “Pink” and “Cerise” respectively, exhibit no rooting ability. On the right—rooted cuttings of the interspecific hybrid DTJ-16-4583.

FIG. 2 is a picture of the parental lines generating a hybrid which is also shown according to some embodiments of the invention.

FIGS. 3A-C are illustrations of various inflorescence types envisaged by some embodiments of the present invention. FIG. 3A—Head type inflorescence; FIG. 3B—Spray type inflorescence; and FIG. 3C—Short spray type inflorescence.

FIG. 4 embodiments for induction of ploidy in the process of breeding.

### DESCRIPTION OF SPECIFIC EMBODIMENTS OF THE INVENTION

The present invention, in some embodiments thereof, relates to interspecific Dianthus plants and methods of producing same.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

In order to generate new varieties of Dianthus which exhibit elite properties, new plants were created as interspecific hybrids of the genus Dianthus by cross-pollination of Dianthus barbatus (D. barbatus), with seedlings of D. japonicus that were selected for early flowering. Selection for early flowering was essential for the success of this interspecific crossing as the two species don't share the same flowering habit. The new interspecific hybrid plants are novel cultivars that combine the best qualities of both the parents. The new hybrids exhibit various features with regard to flower type, flower size, flower color, inflorescence branching, height, rooting and general vigor. Most of the variation in those traits is attributed to the parental species D. barbatus while the broad leaf, foliage type, strong stem, heat tolerance and resistance to Fusarium oxysporum f.sp. dianthi, race 2 is attributed to the parental species D. japonicus.

Surprisingly, despite the lack of scent in both parents, the hybrids are endowed with a sweet scent.

Thus, according to an aspect of the invention there is provided an interspecific Dianthus plant comprising a genomic sequence distinctive of D. barbatus and a genomic sequence distinctive of D. japonicus.

It will be appreciated that although the present disclosure details in great length hybrids of D. barbatus and a genomic sequence distinctive of D. japonicus, embodiments of the invention relate to any interspecific plant, which is the result of crossing with D. japonicus.

Thus, according to another aspect of the invention there is provided an interspecific Dianthus plant comprising a genomic sequence distinctive of D. japonicus and a genomic sequence distinctive of Dianthus species not being D. japonicus, the plant exhibiting resistance to Fusarium oxysporum f.sp. dianthi, race 2 which is higher than that of the Dianthus species.

As used herein, the term “plant” encompasses a whole plant, a grafted plant, ancestor(s) and progeny of the plants and plant parts, including seeds, shoots, stems, roots, rootstock, scion, and plant cells, tissues and organs. The plant may be in any form including suspension cultures, embryos, meristematic regions, callus tissue, leaves, seedlings, cuttings (rooted or unrooted), cut flowers, potted plants, gametophytes, sporophytes and pollen.

As used herein “Dianthus” refers to a genus of about 300 species of flowering plants in the family Caryophyllaceae, native mainly to Europe and Asia.

As used herein the term “an interspecific Dianthus plant” refers to an F1 hybrid progeny of the cross between D. barbatus and D. japonicus.

The D. barbatus and D. japonicus are thus the parents of the interspecific Dianthus plant.

According to a specific embodiment, the interspecific hybrid is not an interspecific hybrid between carnation (Dianthus caryophyllus L.) and Dianthus japonicus Thunb.

Accordingly, the interspecific Dianthus plant is non-transgenic.

The interspecific Dianthus plant comprises genomic sequences of D. barbatus and genomic sequences of D. japonicus. Such genomic sequences may also be referred to as introgression(s).

According to a specific embodiment, some of the genomic sequences distinctive of the parental species are identifiable using standard molecular biology tools, such as used in marker assisted breeding.

As used herein “distinctive” refers to the ability to distinguish between the D. barbatus genome and the D. japonicus genome.

Distinctive sequences can be at least 30 bases long. According to a specific embodiment, the distinctive sequences are SEQ ID NO: 1 and SEQ ID NO: 2 that are specific to D. japonicas and SEQ ID NO: 3 and SEQ ID NO: 4 that are specific to D. barbatus.

According to a specific embodiment, the genomic sequence distinctive of D. barbatus is as set forth in SEQ ID NO: 3 or SEQ ID NO: 4.

According to a specific embodiment, the genomic sequence distinctive of D. japonicus is as set forth in SEQ ID NO: 1 or SEQ ID NO: 2.

The sequence information and annotations uncovered by the present teachings can be harnessed in favor of classical breeding. Thus, sub-sequence data of those polynucleotides described above, can be used as markers for marker assisted selection (MAS) or identification of those plants that are contemplated according to the present teachings i.e., interspecific Dianthus plants. Nucleic acid data of the present teachings (DNA or RNA sequence) may contain or be linked to polymorphic sites or genetic markers on the genome such as restriction fragment length polymorphism (RFLP), microsatellites and single nucleotide polymorphism (SNP), DNA fingerprinting (DFP), amplified fragment length polymorphism (AFLP), expression level polymorphism, polymorphism of the encoded polypeptide and any other polymorphism at the DNA or RNA sequence.

Examples of marker assisted selections include, but are not limited to, selection for a morphological trait (e.g., flower type, coloration, foliage morphology, color, plant height, number of flowers etc.); selection for a biochemical trait; selection for a biological trait (e.g., pathogen resistance, heat tolerance).

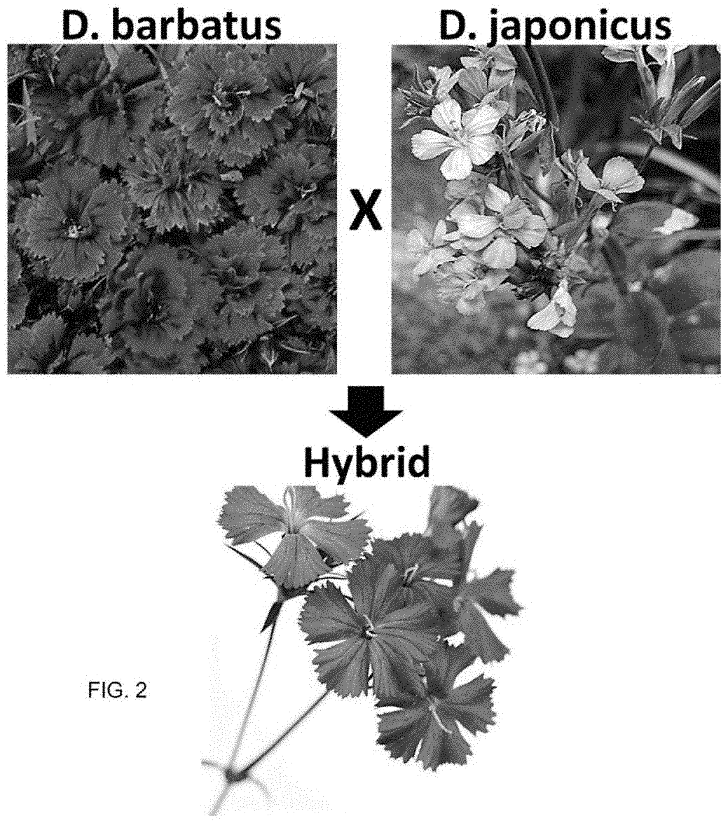

FIG. 2 shows an embodiment of a flowering hybrid according to the present invention.

According to a specific embodiment, the wherein flowers of the interspecific plant are endowed with a sweet scent and/or higher shelf life compared to that of a D. barbatus parent or about the same as that of a D. japonicus parent of the interspecific plant.

According to a specific embodiment, the wherein flowers of the interspecific plant are endowed with higher shelf life compared to that of a D. barbatus parent or about the same as that of a D. japonicus parent of the interspecific plant.

According to a specific embodiment, the wherein flowers of the interspecific plant are not endowed with a sweet scent.

According to some embodiments, the interspecific Dianthus plant is characterized by the average features ±10% (also referred to as “about”) as those described in Table 1 below.

According to another embodiment, “an interspecific Dianthus plant” refers to an F1 hybrid progeny of the cross between D. japonicus and any Dianthus species not being D. japonicus.

Examples of such species include, but are not limited to those listed below:
Dianthus alpinus
Dianthus amurensis
Dianthus anatolicus
Dianthus arenarius
Dianthus armeria
Dianthus balbisii
Dianthus barbatus
Dianthus biflorus
Dianthus brevicaulis
Dianthus burgasensis
Dianthus callizonus
Dianthus campestris
Dianthus capitatus
Dianthus carthusianorum
Dianthus caryophyllus
Dianthus chinensis
Dianthus cruentus
Diyanthus cyprius
Dianthus deltoides
Dianthus erinaceus
Dianthus fragrans
Dianthus freynii
Dianthus fruticosus
Dianthus furcatus
Dianthus gallicus
Dianthus giganteus
Dianthus glacialis
Dianthus gracilis
Dianthus graniticus
Dianthus gratianopolitanus
Dianthus haematocalyx
Dianthus japigicus
Dianthus kladovanus
Dianthus knappii
Dianthus libanotis
Dianthus lusitanus
Dianthus microlepsis
Dianthus moesiacus
Dianthus monspessulanus
Dianthus myrtinervius
Dianthus nardiformis
Dianthus nitidus
Dianthus pavonius
Dianthus pendulus
Dianthus petraeus
Dianthus pinifolius
Dianthus plumarius
Dianthus pungens
Dianthus repens
Dianthus scardicus
Dianthus seguieri
Dianthus simulans
Dianthus spiculifolius
Dianthus squarrosus
Dianthus strictus
Dianthus subacaulis
Dianthus superbus
Dianthus sylvestris
Dianthus tenuifolius
Dianthus urumoffii
Dianthus zonatus

According to some embodiments of the invention, the interspecific plant exhibits resistance to Fusarium oxysporum f.sp. dianthi, race 2 which is higher than that of the non-D. japanicus parent e.g., D. barbatus parent or at least about the same as that of the D. japonicus parent of the interspecific plant.

Fusarium oxysporum f.sp. dianthi refers to a fungal plant pathogen infecting species of the genus Dianthus.

One of the most severe problems affecting carnation (Dianthus caryophyllus L.), in most areas of the world where the crop is grown, is the soilborne pathogen fungus Fusarium oxysporum forma specialis dianthi (Fod) race 2 (Holley and Baker, 1991). The pathogen causes vascular wilt of carnation, a disease called Fusarium wilt. This disease causes extensive yield loss; symptoms consist of yellowing and wilting of branches that gradually progress until the plant dies, leading to deterioration in quality and quantity of marketable blooms and planting materials.

A new source of resistance to Fusarium oxysporum f.sp. dianthi, race 2, was discovered in D. japonicus and had been transmitted by inheritance to the interspecific hybrids. Artificial inoculation with the pathogen, as a bioassay, followed by phenotypic scoring can be conducted in order to determine resistance. Results are presented in Table 4. Varieties of D. japonicas DTJ-16-4787 and DTJ-16-4788 that were used as male parents in the cross to produce the interspecific hybrid according to some embodiments of the invention, are highly resistant to Fusarium oxysporum f.sp. dianthi, race 2, while the maternal varieties of D. barbatus, DT-Z-171, DT-Z-173 and DT-Z-174, display variable level of sensitivity. All the interspecific hybrids tested showed a very high level of resistance.

The Examples section which follows provides details of a bioassay for determining tolerances to infection by Fusarium oxysporum f.sp. dianthi, race 2.

According to a specific embodiment, the interspecific plant exhibits increased tolerance of at least 10 fold (e.g., 13, 15, 20, 50, 100) as compared to that of D. barbatus.

According to a specific embodiment, interspecific dianthus plants exhibit 100% survival following infection with the pathogen.

According to a specific embodiment, interspecific dianthus plants exhibit resistance to Fusarium oxysporum f.sp. dianthi, race 2, which is about the same as that of D. japonicus.

Resistance to the pathogen can be determined by scoring infection symptom severity from healthy to dead plants, as shown in Table 4 of the Examples section which follows.

According to a specific embodiment, resistance also refers to prevention of infection by the pathogen.

According to an additional or an alternative embodiment of the invention, the interspecific plant exhibits heat tolerance which is at least about the same or higher than that of a D. japonicus parent of the interspecific plant.

As used herein “higher heat tolerance” refers to less wilting and less detrimental effects on flower quality (petal burn, fewer petals, smaller flowers) as compared to that of the D. barbatus parent.

For comparison the hybrid is endowed with higher heat tolerance than that of D. caryophyllus which does not respond by wilting at temperatures up to 35 degrees Celsius, rather the stem and flower quality degenerate: as manifested by small flowers, hollow flowers, thin stems, flower and/or leaf burn.

In comparison the interspecific hybrids show some mild wilting at midday but flower and stem quality is much less affected than those of D. caryophyllus and remain good. Heat tolerance is important in breeding Dianthus, since varieties which continue flowering with quality stems throughout the summer season, enable the growers to extend their season and get more yield per square meter. D. japonicus exhibits unusually high tolerance to high temperature: whereas, D. barbatus begins to show significant heat stress at 30° C., D. japonicus only shows mild symptoms at temperatures nearing 40° C. The interspecific hybrids selected according to some embodiments of the invention display a phenotypic appearance which is about the same as that of the parental species D. japonicas with regard to heat tolerance.

According to an additional or an alternative embodiment of the invention, the interspecific plant exhibits a flower size which is smaller (e.g., at least by 2%, 3%, 4%, 5%, 6%, 7%, 8%, 9% 10%, 11%, 15%, 18%, 20%) than that of a D. barbatus parent or larger than that of a D. japonicus parent (e.g., by at least 5%, 10%, 15%, 20% 25%, 30%) of the interspecific plant.

According to a specific embodiment the average flower size is: 1-6 cm, 1-5 cm, 1.5-6 cm, 1.5-5 cm, 1.5-4 cm; 1.5-3.8 cm; 1.5-3.6 cm; 1.5-3.4 cm; 1.5-3.2 cm; 1.5-3 cm; 1.5-2.8 cm; 1.5-2.6 cm; 1.5-2.4 cm; 1.5-2.2 cm; 1.5-2 cm; 1.5-1.8 cm; 1.5-1.6 cm; 1.6-3.1 cm; 1.7-3.1 cm; 1.8-3.1 cm; 1.9-3.1 cm; 2-3.1 cm; 2.1-3.1 cm; 2.2-3.1 cm; 2.3-3.1 cm; 2.4-3.1 cm; 2.5-3.1 cm; 2.6-3.1 cm; 2.7-3.1 cm; 2.8-3.1 cm; 2.9-3.1 cm, 3-6 cm, 3-5 cm, 3-4 cm.

According to an additional or an alternative embodiment of the invention, the interspecific plant exhibits a stem length of 15-120 cm.

According to an additional or an alternative embodiment of the invention, the interspecific plant exhibits a stem length of at least 60 cm (e.g., at least 62 cm, 64 cm, 66 cm, 68 cm, 70 cm, 72 cm, 74 cm, 76 cm, 78 cm, 80 cm, 82 cm, 84 cm, 86 cm, 88 cm, 90 cm, 92 cm, 94 cm, 96 cm, 98 cm, 100 cm, 102 cm, 104 cm, 106 cm, 108 cm, 110 cm, 112 cm, 114 cm, 116 cm, 118 cm 120 cm, 125 cm, 130 cm; e.g., 60-100 cm, 60-130 cm, 62-120 cm). Such a stem can be used for cut flowers.

According to an additional or an alternative embodiment of the invention, the interspecific plant exhibits a stem length of less than 60 cm (e.g., less than 55 cm, 50 cm, 45 cm, 20 cm). This shorter range can be used in bedding.

According to an additional or an alternative embodiment of the invention, the interspecific plant exhibits a number of stems per plant which is at least as that of a D. barbatus parent or a D. japonicus parent of the interspecific plant.

According to a specific embodiment, the interspecific plant exhibits a number of stems equal to or greater than either of the parent species. Examples include, but are not limited to 3-30, 10-30, 10-25, 10-20, 10-15.

Accordingly, the number of stems per plant is higher than 9, e.g., higher than 10, 11, 12, 13, 14, 15, 16, 17, 18, 19, 20, 21, 22, 23, 24 25, 30.

According to an additional or an alternative embodiment of the invention, the interspecific plant exhibits a single flower type.

According to an additional or an alternative embodiment of the invention, the interspecific plant exhibits a double flower type.

According to an additional or an alternative embodiment of the invention, the interspecific plant exhibits a semi-double flower type.

A single flower contains one whorl of sepals, one whorl of Petals the sex organs of the flower remain intact (anthers, pistil, ovary).

Semi-double flowers in Dianthus are characterized by more than one whorl of petals. However the sex organs of the flower remain intact (anthers, pistil, ovary).

In fully double flowers, also referred to as fully-double flowers, the anthers are converted to petaloids and the pistil is often underdeveloped.

According to an additional or an alternative embodiment of the invention, the interspecific plant exhibits a flower color selected from the group consisting of dark pink, cherry, light pink, purple and cream white.

According to an additional or an alternative embodiment of the invention, the interspecific plant exhibits a flower color selected from the group consisting of yellow, white, orange, shades of pink, red, burgundy, very dark burgundy and purple.

According to an additional or an alternative embodiment of the invention, the interspecific plant exhibits a flower color, which is a combination of any of the aforementioned colors.

As used herein “dark pink” refers to an RSH code selected from the group consisting of 59B, N74A, 77B, 64C, 71C, RHS 67B.

As used herein “CERISE” refers to an RSH code selected from the group consisting of 67B, 64C, 68A, N74A, 64C, N66D, N66C, 70B, N66B, 86B, 73A, N57 B+C, 77C.

As used herein “light pink” refers to an RSH code selected from the group consisting of 69D/73B, 69D/62C, 65C, 69B/N66D, 75A.

As used herein “cream white” refers to an RSH code 155B.

As used herein “purple” refers to an RSH code RHS 81A.

According to an additional or an alternative embodiment of the invention, the interspecific plant exhibits a flower pattern selected from the group consisting of plain, vein and bicolor.

According to a specific embodiment, the interspecific plant does not flower but rather exhibits a green flower-like structure. In such a case flower development is blocked prior to the formation of the perianth resulting in green tufts of leaves consisting primarily of bracts. DTJ-18-6042, is an exemplary hybrid of such an interspecific plant.

According to an additional or an alternative embodiment of the invention, the interspecific plant exhibits an inflorescence type selected from the group consisting of a head type inflorescence and a spray type inflorescence.

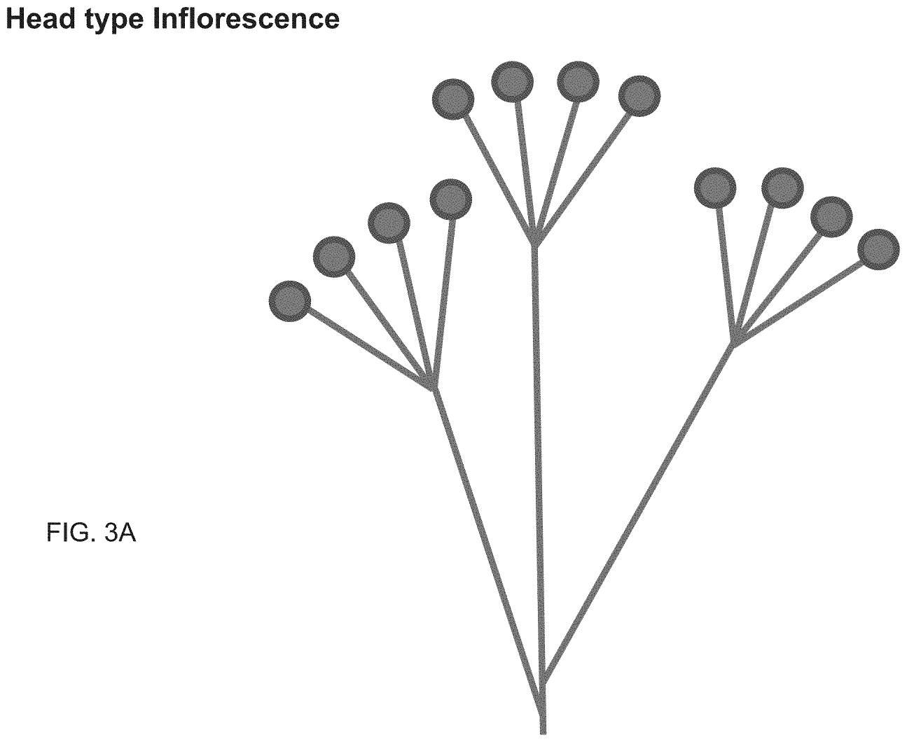

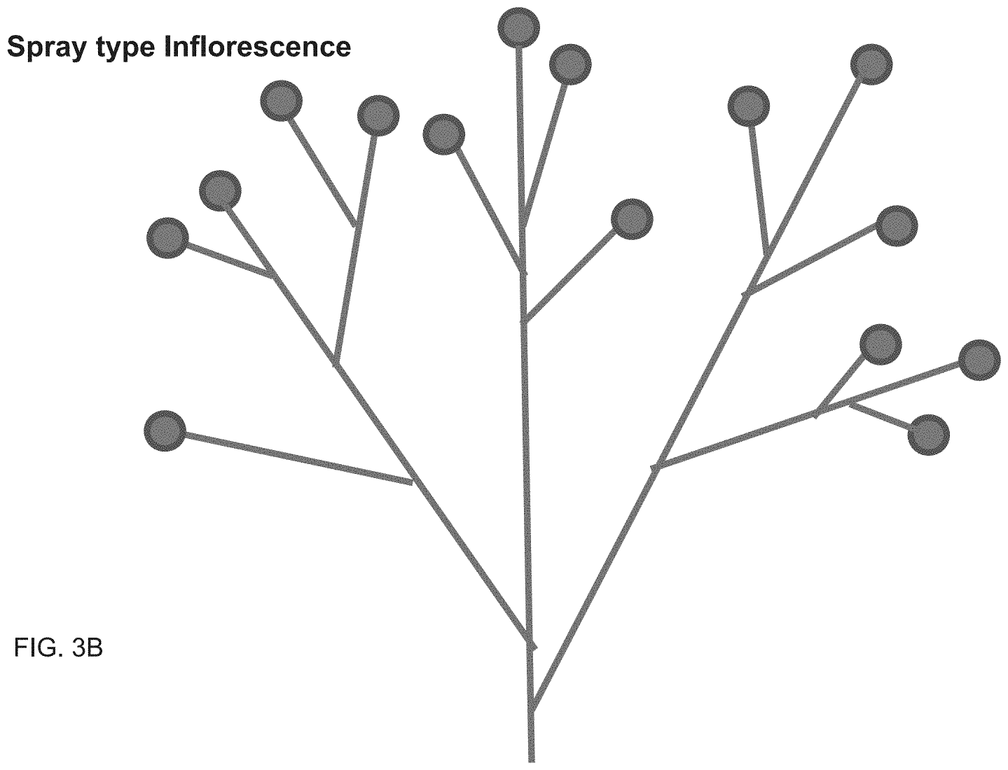

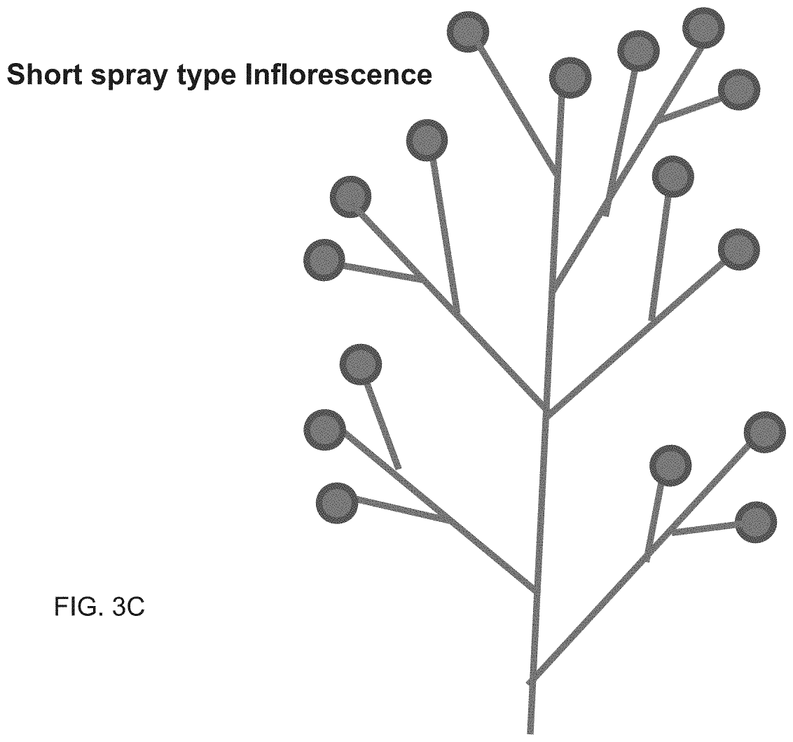

FIGS. 3A-C are illustrations of various inflorescence types envisaged by some embodiments of the present invention.

A head-type inflorescence which is characterized in that the flowers are attached by pedicels directly to the main stem forming a ball shaped cyme at the apex of the stem.

A spray-type inflorescence, which is characterized in that the flowers are attached to the main stem by long peduncles starting midway up the stem.

A short spray type inflorescence, which is similar to the spray type only the peduncles are much shorter.

According to a specific embodiment the foliage length is longer by e.g., at least by 2%, 5%, 10%, 15%, 20%, 30%, 50%, 75%, 100%, 120%, 150%, 180%, 200%, 225%, 250% 275% 300%, 350%, 400%, 500%; e.g., 9-30 cm, 10-30 cm, 11-30, 12-30 cm, 13-30 cm, 14-30 cm, 15-30 cm, 16-30 cm, 17-30 cm, 18-30 cm, 19-30 cm, 20-30 cm, 21-30 cm, 22-30 cm, 23-30 cm, 24-30 cm, 25-30 cm, 26-30 cm, 27-30 cm, 28-30 cm.

According to some embodiments of the invention, the interspecific plant exhibits rooting ability at least as good as that of a D. barbatus parent of the interspecific plant.

As used herein “rooting ability” is defined as percentage of rooted cuttings. According to a specific embodiment, at least 20% of the cuttings are rooted e.g., at least 30%, 35%, 40%, 45%, 50%, 55%, 60%, 65%, 70%, 75%, 80%, 85%, 90%, 95% or even 100%.

According to some embodiments of the invention, the interspecific plant is sterile.

According to some embodiments of the invention, the interspecific plant is fertile.

According to some embodiments of the invention, a sample of representative seeds of a D. Japonicus parent of the interspecific plant has been deposited under the Budapest Treaty at the NCIMB under NCIMB 43450 (DTJ-16-4748) on Aug. 2, 2019.

According to an aspect, the interspecific Dianthus plant is obtainable by crossing (classical breeding).

Accordingly there is provided a method of producing the interspecific Dianthus plant, the method comprising:

(a) selecting from a plurality of D. japonicus plants an early flowering D. japonicus plant;

(b) crossing the early flowering D. japonicus plant with a D. barbatus plant so as to obtain the interspecific Dianthus plant.

Alternatively or additionally, there is provided a method of producing the interspecific Dianthus plant as described herein, the method comprising:

(a) selecting from a plurality of D. japonicus plants an early flowering D. japonicus plant;

(b) crossing the early flowering D. japonicus plant with a Dianthus species not being D. japonicus so as to obtain the interspecific Dianthus plant.

As used herein “early flowering” refers to at least about 3 or 4 weeks earlier than the typical botanical Japonicus.

In order to cross the plants efficiently it is imperative to select for matching flowering time. D. barbatus flowers in Israel early in the spring time (February to May) while D. japonicus flowers late in the summer time (June to July). While D. japonicus flower late in the summer season, the fertility of D. barbatus decrease significantly as the temperature increase during the summer. In order to select for seedling of D. japonicus that are early flowering the inventor germinated about 200 seedlings and planted them between January to March 2015 on heated table in the greenhouse (average day temperature 20-30° C.). The inventor selected lines, which were the ones which flowered (e.g., DTJ-16-4787 and DTJ-16-4788). These can be the subject for crossing with D. barbatus.

In order to increase variation in D. japonicus, the method further comprises mutagenesis (i.e., subjecting to a mutagen) of the early flowering D. japonicus plants prior to the crossing.

The mutagen can be a chemical or physical mutagen (as described in the Examples section). Examples of chemical mutagens include, but are not limited to nitrous acid, alkylating agents such as ethyl methanesulfonate (EMS), methyl methane sulfonate (MMS), diethylsulfate (DES), and base analogs such as 5-bromo-deoxyuridine (5BU). Physical mutagens include radiation (e.g. fast neutron, x-ray, gamma radiation).

According to a specific embodiment, radiating comprises gamma radiation. Irradiated lines with beneficial properties are described in the Examples section, which follows.

As the flowers of D. japonicus are relatively small and emasculation is difficult, the male parent in the method of breeding is selected D. japonicus while the female parent is selected D. barbatus.

Following crossing the method further comprises selecting for at least one of the following traits:

single or double flower type;

height of at least 60 cm;

height below 60 cm;

attractive and durable foliage;

dark, wide, shiny leaves typical of D. japonicus (e.g., RHS139A);

Number of stems per plant of at least 4 e.g., at least 6, and intense basal branching simultaneously;

heat tolerance higher than that of D. caryophyllus. and optionally than that of D Japonicus; and

resistance to Fusarium oxysporum f.sp. dianthi, race 2 at least as good as that of the D. japonicus parent.

Specific embodiments of such selection criteria are described herein.

It will be appreciated that any time during the breeding process, the parent(s) and/or the hybrid (e.g., F1), or during backcrossing the plant can be subject to induction of polyploidy.

Polyploidy may facilitate in genome buffering and increasing allelic diversity. Alternatively or additionally, polyploidy may contribute to the heterozygosity and increase the probability of heterosis in plants' progenies, as well as increase fertility.

As used herein “polyploidy” refers to the addition of at least one chromosome or more compared to the native polidy of the plant. D. japonicus and D. barbatus are both diploid in nature (n=15, 2n=30). Any addition beyond 30 is considered a polyploidy. According to a specific embodiment, the addition is of a complete chromosome set resulting in a triplid, tetraploid, pentaploid, hexaploid etc. plant.

According to a specific embodiment, the plant is triploid.

According to a specific embodiment, the plant is tetraploid.

The duplication may be induced in the parent generation so as to produce polyploid gametes; in the hybrid progeny or in the process of the interspecific cross or back-crossing. Genome duplication can occur in already polyploid organism.

The term “polyploidy” may encompass various forms as described above and further defined hereinbelow.

As used herein “euploid” refers to a polyploidy with exact multiples of the complete set of chromosomes specific to a species. Haploid or Monoploid (one set; 1n), Diploid (two sets; 2n), Triploid (three sets; 3n), Tetraploid (four sets; 4n), Pentaploid (five sets; 5n), Hexaploid (six sets; 6n), Octaploid (eight sets; 8n), Decaploid (ten sets; 10n), Dodecaploid (12 sets; 12n).

As used herein “autopolyploid” refers to a polyploid with multiple chromosome sets derived from a single species—usually derived from chromosome doubling.

As used herein “allopolyploid” refers to a polyploid whose chromosomes are derived from different species, are a combination of genomes of different species due to hybridization of two or more genomes followed by chromosome doubling or by the fusion of unreduced gametes between species.

As used herein “aneuploidy” refers to a polyploid that contains either an addition or deletion of one or more specific chromosome(s) to the total number of chromosomes that usually make up the ploidy of a species (Acquaah 2007; Ramsey and Schemske 1998). Aneuploids are formed due to the formation of univalents and multivalents during meiosis of euploids.

Each of these configurations is contemplated herein.

It will be appreciated that ploidy can be induced chemically or physically (e.g., irradiation e.g., see above and in Example 7 which follows).

Induction of polyploidy is typically performed by subjecting a plant tissue to a G2/M cycle inhibitor.

Typically, the G2/M cycle inhibitor comprises a microtubule polymerization inhibitor.

Examples of microtubule cycle inhibitors include, but are not limited to oryzalin, colchicine, colcemid, trifluralin, benzimidazole carbamates (e.g. nocodazole, oncodazole, mebendazole, R 17934, MBC), o-isopropyl N-phenyl carbamate, chloroisopropyl N-phenyl carbamate, amiprophos-methyl, taxol, vinblastine, griseofulvin, caffeine, bis-ANS, maytansine, vinbalstine, vinblastine sulphate and podophyllotoxin.

According to a specific embodiment, the microtubule cycle inhibitor is colchicine.

According to a specific embodiment, the microtubule cycle inhibitor is oryzalin.

According to a specific embodiment, the polyploidy inducer is an alkylating agent, hydroxylamine NH₂OH, base analogues, agents that form DNA adducts, DNA intercalating agents, DNA crosslinkers, oxidative damage inducers and nitrous acids.

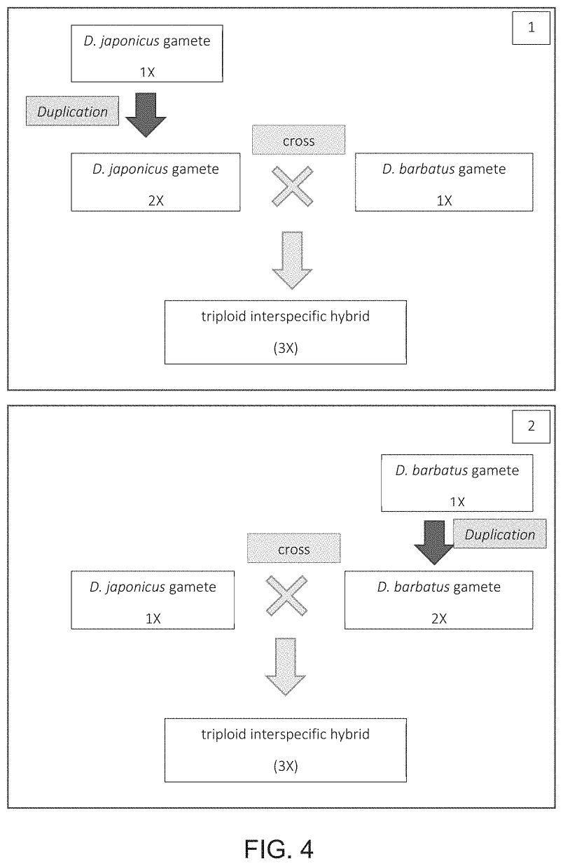

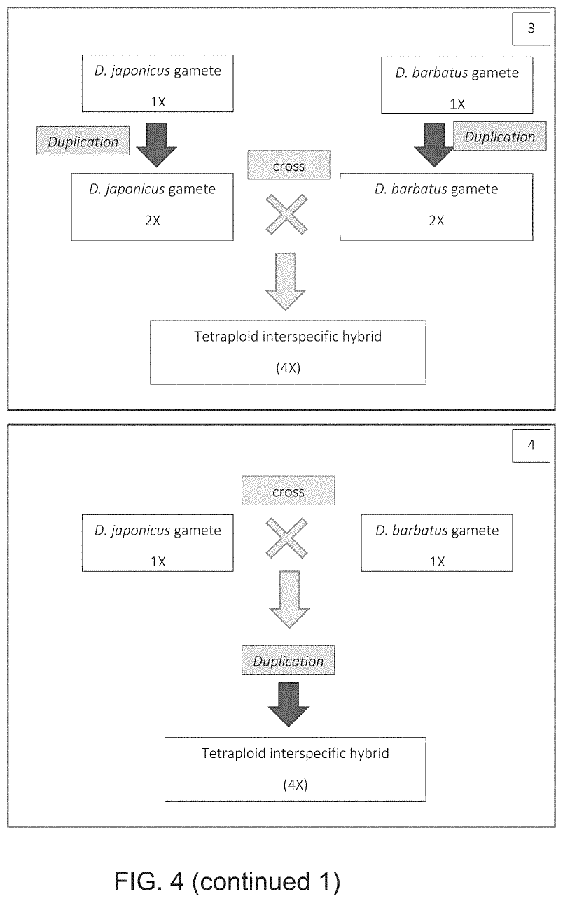

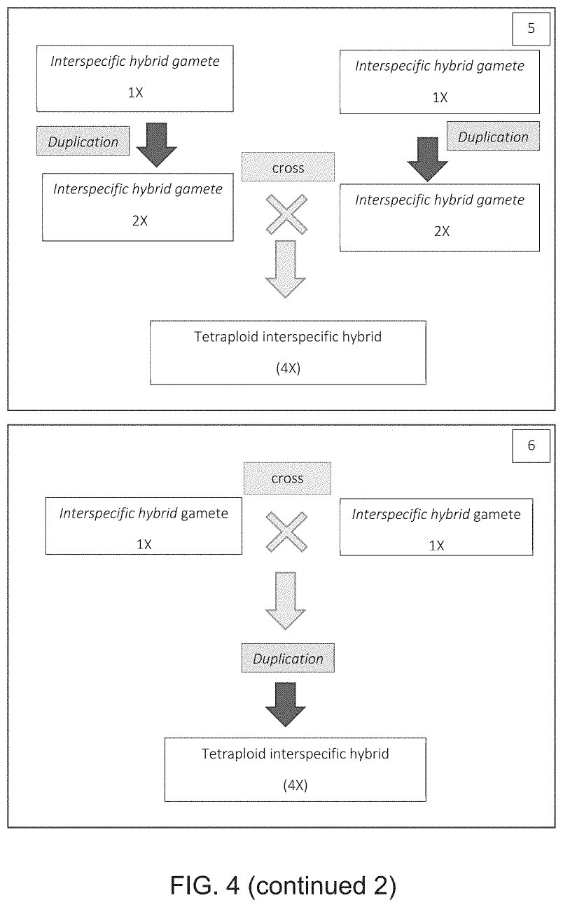

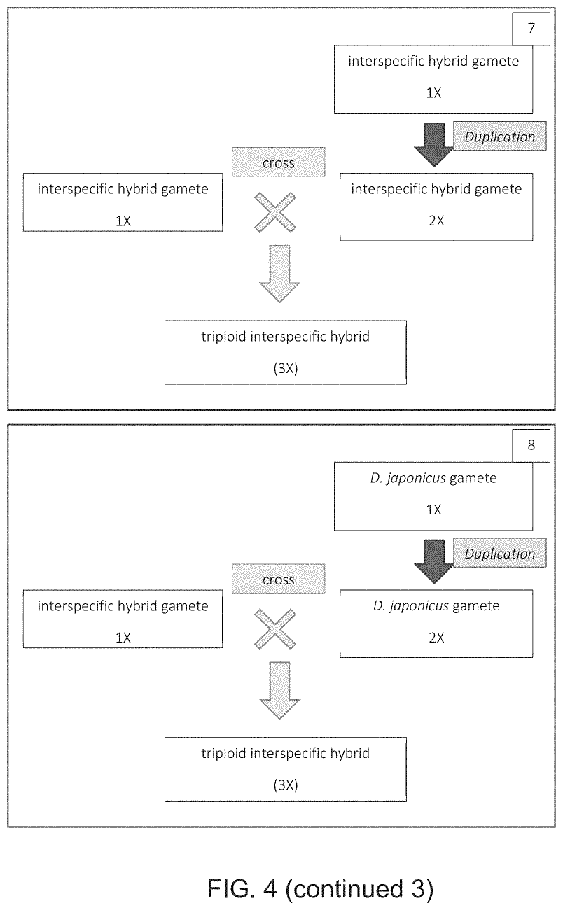

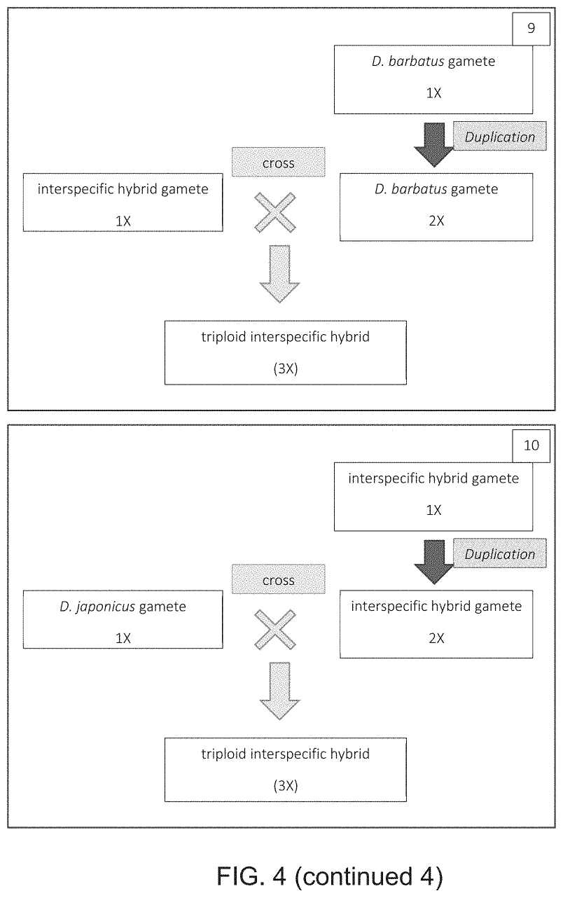

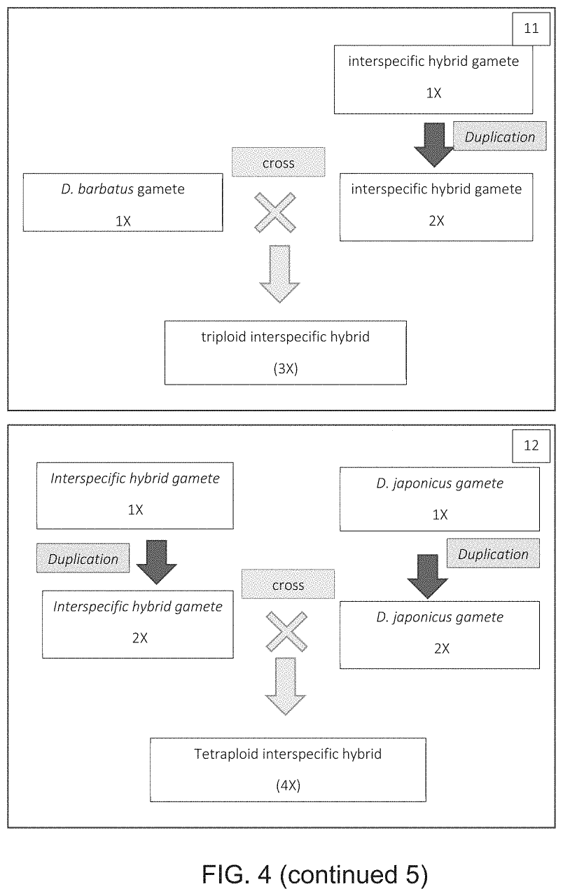

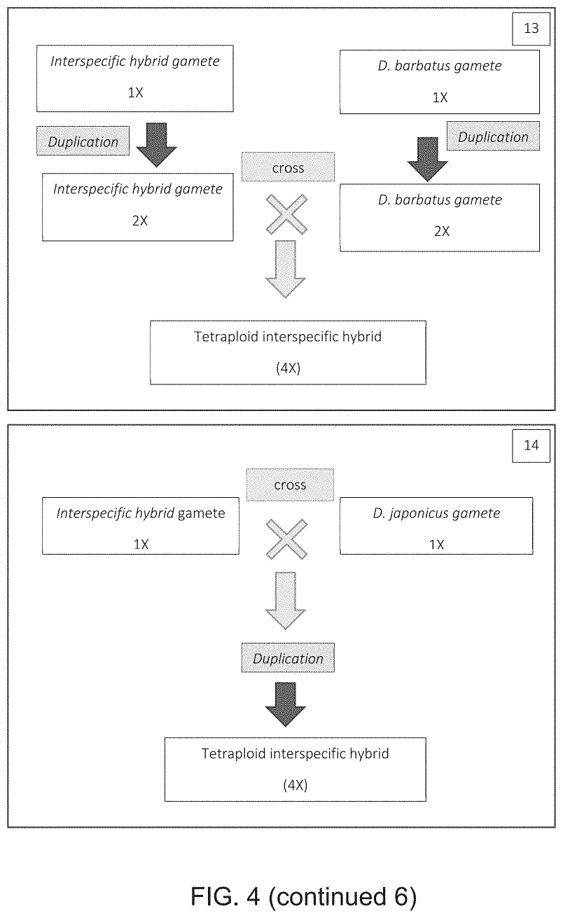

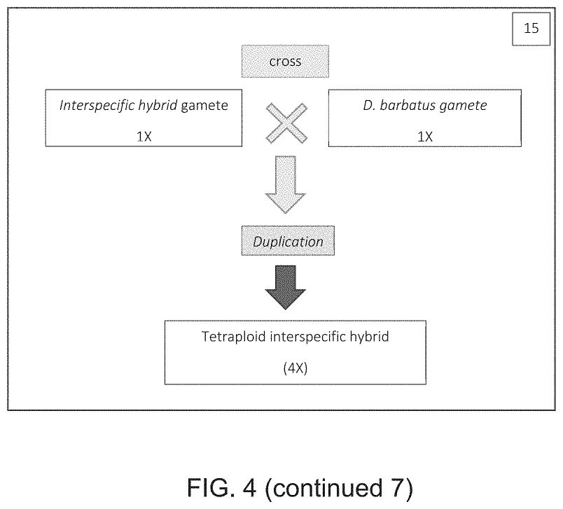

FIG. 4 describes non-limiting examples of the induction of polyploidy during the breeding process. A brief description of each is provided infra.

Polyploidy induced in the one or more of the interspecific parents can be described as follows:

1. [D. japonicus gamete (1×)+duplication]*D. barbatus gamete (1×)=D. japonicus gamete (2×)*D. barbatus gamete (1×)=triploid interspecific hybrid zygote (3×)

2. D. japonicus gamete (1×)*[D. barbatus gamete (1×)+duplication]=D. japonicus gamete (1×)*D. barbatus gamete (2×)=triploid interspecific hybrid zygote (3×)

3. [D. japonicus gamete (1×)+duplication]*[D. barbatus gamete (1×)+duplication]=D. japonicus gamete (2×)*D. barbatus gamete (2×)=tetraploid interspecific hybrid zygote (4×)

Polyploidy Induced in the Process of Cross (after Zygote) to Create the Interspecific Hybrid can be Described as Follows:

4. D. japonicus gamete (1×)*D. barbatus gamete(1×)=F1−interspecific hybrid zygote (2×)+duplication=tetraploid interspecific hybrid (4×)

Polyploidy Induced in Interspecific Hybrid can be Described as Follows:

5. [Interspecific hybrid gamete (1×)+duplication]*[Interspecific hybrid gamete (1×)+duplication]=Interspecific hybrid gamete (2×)*Interspecific hybrid gamete (2×)=tetraploid interspecific hybrid zygote (4×)

6. Interspecific hybrid gamete (1×)*Interspecific hybrid gamete (1×)=diploid interspecific hybrid zygote (2×)+duplication=tetraploid interspecific hybrid (4×)

7. [Interspecific hybrid gamete (1×)+duplication]*Interspecific hybrid gamete (1×)=Interspecific hybrid gamete (2×)*Interspecific hybrid gamete (1×)=triploid interspecific hybrid (3×)

Polyploidy induced during back-cross of interspecific hybrids with D. japonicus or D. barbatus can be described as follows:

Duplication Induced in Patents:

8. Interspecific hybrid gamete (1×)*[D. japonicus gamete (1×)+duplication]=Interspecific hybrid gamete (1×)*D. japonicus gamete (2×)=triploid interspecific hybrid zygote (3×)

9. Interspecific hybrid gamete (1×)*[D. barbatus gamete (1×)+duplication]=Interspecific hybrid gamete (1×)*D. barbatus gamete (2×)=triploid interspecific hybrid zygote (3×)

10. [Interspecific hybrid gamete (1×)+duplication]*D. japonicus gamete (1×)=Interspecific hybrid gamete (2×)*D. japonicus gamete (1×)=triploid interspecific hybrid (3×)

11. [Interspecific hybrid gamete (1×)+duplication]*D. barbatus gamete (1×)=Interspecific hybrid gamete (2×)*D. barbatus gamete (1×)=triploid interspecific hybrid (3×)

12. [Interspecific hybrid gamete (1×)+duplication]*[D. japonicus gamete (1×)+duplication]=Interspecific hybrid gamete (2×)*D. japonicus gamete (2×)=tetraploid interspecific hybrid zygote (4×)

13. [Interspecific hybrid gamete (1×)+duplication]*[D. barbatus gamete (1×)+duplication]=Interspecific hybrid gamete (2×)*D. barbatus gamete (2×)=tetraploid interspecific hybrid zygote (4×)

Duplication Induced in the Process of Crossing (after Zygote):

14. Interspecific hybrid gamete (1×)*D. japonicus gamete (1×)=diploid interspecific hybrid zygote (2×)+duplication=tetraploid interspecific hybrid (4×)

15. Interspecific hybrid gamete (1×)*D. barbatus gamete (1×)=diploid interspecific hybrid zygote (2×)+duplication=tetraploid interspecific hybrid (4×)

Accordingly, in an embodiment of the invention, the breeding process comprises subjecting said D. japonicus plants and/or said D. barbatus plant or a portion thereof to a polyploidy induction treatment.

According to an additional or alternative embodiment, the method comprises subjecting said interspecific Dianthus plant or portion thereof to a polyploidy induction treatment.

According to an additional or alternative embodiment, the method comprises back-crossing said interspecific Dianthus plant to a recurrent parent.

According to an additional or alternative embodiment, the method comprises subjecting said interspecific Dianthus plant or said recurrent parent or portion thereof to a polyploidy induction treatment.

According to a specific embodiment, ploidy is induced after the interspecific cross to restore fertility of the hybrid.

According to a specific embodiment, the plant material that is subjected to polyploidy induction treatment is grown and closely observed. Pollen from the treated plants is collected and applied on other treated plants and on non-treated plants.

Progeny of all treated plants is examined as well. According to a specific embodiment, phenotypic selection takes place to identify the polyploid varieties. Plants presenting an abnormal phenotype are closely examined.

For example, plants presenting low vigor and/or low vitality are suspected of suffering from mutations, primarily deletion of essential genes or chromosomes.

Plants presenting higher vigor, larger organs, different plant structure, smaller organs, unusual color or color pattern, change in fragrance, different flower structure compared to the parents etc. are further examined and samples of their tissue are collected for chromosomal analysis such as by flow cytometry or sequencing.

According to a specific embodiment, there is provided hybrid seed obtainable according to embodiments described herein.

Thus, the present teachings refer to interspecific Dianthus plant or its parts, including cut flowers, produced by growing seed of the present invention. In one embodiment, the plant is used in combination mix containers.

Also provided is a tissue culture of the interspecific plant described herein.

Also provided is a potted flower the interspecific plant described herein.

According to a specific embodiment, the plant is propagated by vegetative means.

According to some embodiments of the invention, seeds of a parental D. japanicus forming hybrids as described herein have been deposited under the Budapest Treaty at the NCIMB under NCIMB 43450 (DTJ-16-4748) on Aug. 2, 2019.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Example 1

### Parent Plants and the Production of the Interspecific Hybrids

Embodiments of the invention relate to novel plants with unique characteristics and attractive flowers, desirable for cut flower and bedding plants. The new plants are interspecific hybrids, created in Nir Zvi, Israel. The hybrids are the result of a cross done by the inventor after a selection of seedlings to eliminate differences in flower timing of the two parental species. D. barbatus flowers early in the spring time (February to May in Israel), while D. japonicus flower late in the summer time (June to July in Israel).

While D. japonicus flower late in the summer season, the fertility of D. barbatus decreases significantly as the temperature increases during the summer.

In order to select for seedlings of D. japonicus that are early flowering, 200 seedlings were germinated and planted between January to March 2015 on a heated table in the greenhouse (average day temperature 20-30° C.). Two new lines were selected, which were the only ones who flowered. These were designated as DTJ-16-4787 and DTJ-16-4788 with flower colors of pink and cerise, respectively. Due to the self-pollinating nature of D. japonicus and its small flowers (difficult to emasculate), the selected varieties of D. japonicus were used as the pollen parent and D. barbatus varieties designated DT-Z-171, DT-Z-173 and DT-Z-174 as the maternal parent.

D. japonicus is self-pollinated and has little phenotypic variance in Si progenies. In order to generate genetic variation, mutations were induced by exposure of seeds to gamma radiation. Germination rate was 60%.

After the radiation treatment, 7 new lines were selected out of the plants that survived. The lines were designated DTJ-16-4789, DTJ-16-4790, DTJ-16-4792, DTJ-16-4793, DTJ-16-4794, DTJ-16-4795 and DTJ-16-4796. Line DTJ-16-4794 was selected for early flowering. It flowered 7-14 days (depending on the season) prior to its source DTJ-16-4787. Line DTJ-16-4792 was selected for its new flower color Magenta. Table 1 below, lists the different parents and hybrids.

### Example 2

### Description of the Hybrids Plants

The present invention relates to new and distinctive Dianthus interspecific hybrids. More particularly, it concerns interspecific hybrids plants derived from the cross of D. japonicus with D. barbatus species. The new hybrids selected are characterized by the best characteristics of both the parents' species and display much variation with regard to flower type, flower size, flower color, inflorescence branching, foliage type, height and general vigor. All the interspecific hybrids are sterile and the ones selected are characterized by:

1. Single or double flower type.
2. Sufficient height (>=60 cm) to be used as a cut flower or shorter segregates to be used as bedding plants
3. Attractive and durable foliage; dark, wide, shiny leaves.
4. Good vegetative basal branching.
5. Generally robust habit.
6. Heat tolerance
7. Strong resistance to Fusarium oxysporum f.sp. dianthi, race 2.

From the segregating F1 progeny plants were isolated for a significant percentage (˜10%) that looked to have commercial and/or further breeding potential.

Over the course of the breeding program new hybrids were selected for their novel phenotype and were further evaluated to meet standard agronomic demands, such as ample vegetative growth, rooting ability, flower stem yield and vase life. Phenotypic observations are detailed in Table 1, below.

TABLE 1
Diverse phonotypes observed at the interspecific hybrids.

| | | | | Average | | | | Num- | | Num- | | | | |
|---|---|---|---|---|---|---|---|---|---|---|---|---|---|---|
| | | | | Flower | | | | ber | | ber | | | | |
| | | | | Size | | | | flow- | | of | | | Fo- | |
| | | | Flower | (Diam- | | Average | Flower | ers | Stem | Stems | Foliage | | liage | fo- |
| | | Flower | color | eter, | Flower | Number | Color | on | Height | per | color | Foliage | length | liage |
| Code | Genus | color | (RSH) | cm) | Type | of petals | Pattern | stem | (cm) | plant | (RSH) | Shape | (cm) | width |
| DTJ-16-4788 | D. japonicus | Light | 77C | 1.5 | Single | 5 | Plain | 84 | 85 | 6 | 137 A | Eliptic | 7.5 | |
| | “Cerise” | purple | | | | | | | | | | | | |
| DTJ-16-4787 | D. japonicus | Pink | N 137 B | 1.5 | Single | 5 | Plain | 44 | 85 | 7 | 137 B | Eliptic | 8 | |
| | “Pink” | | | | | | | | | | | | | |
| DT-Z-171 | D. barbatus | Red | 53A | 2.5 | Double | 16 | Vein | 4 | 50 | 7 | N 137 B | Lanceolate | 8 | |
| DT-Z-173 | D. barbatus | Dark | 59 B | 3.2 | Double | 17 | Vein | 10 | 50 | 9 | N 137 B | Lanceolate | 5.5 | |
| | | pink | | | | | | | | | | | | |
| DT-Z-174 | D. barbatus | Purple | 72 A | 3.5 | Double | 18 | Vein | 13 | 54 | 4 | 143 B | Lanceolate | 5.5 | |
| DTJ-16-4808 | Interspecific | Purple | N74 A | 2.2 | Single | 5 | Plain | 76 | 75 | 7 | N137 B | Lanceolate | 11 | 1.5 |
| | hybrid | | | | | | | | | | | | | |
| DTJ-17-5301 | Interspecific | Cream | NN155A | 3.3 | Double | 19.6 | Bicolor | 16 | 71 | 9 | N137 C | Lanceolate | 12 | |
| | hybrid | white | | | | | | | | | | | | |
| DTJ-17-5302 | Interspecific | Light | 69B/ | 2.6 | Double | 20.3 | Vein | 14 | 90 | 2 | 139A | Lanceolate | 19 | 1.5 |
| | hybrid | Pink | N66 D | | | | | | | | | | | |
| DTJ-17-5303 | Interspecific | Cherry | 67B | 1.5 | Double | 16 | Vein | 29 | 36 | 10 | 137C | Lanceolate | 23 | 1.5 |
| | hybrid | | | | | | | | | | | | | |
| DTJ-17-5304 | Interspecific | cherry- | 70B | 2 | Double | 19.6 | Vein | 6 | 90 | 8 | N137 C | Lanceolate | 23 | 1 |
| | hybrid | dark | | | | | | | | | | | | |
| | | pink | | | | | | | | | | | | |
| DTJ-17-5305 | Interspecific | light | 69D/73B | 2.4 | Single | 5 | Plain | 21 | 60 | 13 | N137 C | Lanceolate | 17 | 1 |
| | hybrid | pink/ | | | | | | | | | | | | |
| | | cream | | | | | | | | | | | | |
| | | white | | | | | | | | | | | | |
| DTJ-17-5306 | Interspecific | Cherry | 64C | 2.2 | Single | 5 | Plain | 19 | 80 | 13 | 139A | Lorate | 19 | |
| | hybrid | | | | | | | | | | | | | |
| DTJ-17-5308 | Interspecific | Pink | 73A | 2.3 | Single | 5 | Plain | 8* | 80 | 17 | 137A | Lanceolate | 12 | 1 |
| | hybrid | | | | | | | | | | | | | |
| DTJ-17-5309 | Interspecific | Dark | 71C | 2.6 | Double | 22.6 | Vein | 25 | 90 | 11 | N137 C | Lincar | 18 | |
| | hybrid | pink | | | | | | | | | | | | |
| DTJ-17-5310 | Interspecific | Cherry | 64C | 2 | Double | 19.6 | Vein | 37 | 90 | 12 | N137 A | Lanceolate | 19 | 2 |
| | hybrid | | | | | | | | | | | | | |
| DTJ-17-5311 | Interspecific | Cherry | 67B | 2.5 | Double | 20.6 | Plain | 27 | 68 | 16 | N137 B | Lincar | 19 | |
| | hybrid | | | | | | | | | | | | | |
| DTJ-17-5312 | Interspecific | Cherry | 64C | 2 | Double | 17 | Vein | 29 | 78 | 18 | N137 A | Lanceolate | 21 | 1.5 |
| | hybrid | | | | | | | | | | | | | |
| DTJ-17-5314 | Interspecific | Cherry | 64C | 2 | Single | 5 | Vein | 15* | 90 | 10 | N137 A | Lanceolate | 17 | |
| | hybrid | | | | | | | | | | | | | |
| DTJ-17-5315 | Interspecific | Light | 69D/62C | 1.7 | Single | 5 | Vein | 51 | 68 | 15 | N137 A | Lanceolate | 17.5 | 1 |
| | hybrid | Pink | | | | | | | | | | | | |
| DTJ-17-5316 | Interspecific | Cherry | 64C | 2.3 | Single | 5 | Vein | * | 115 | 8 | N137 B | Lanceolate | 16.5 | 0.6 |
| | hybrid | | | | | | | | | | | | | |
| DTJ-17-5317 | Interspecific | Cherry | 73A | 2.8 | Double | 23.6 | Vein | 5* | 96 | 13 | N137 B | Lanceolate | 16 | 0.5 |
| | hybrid | | | | | | | | | | | | | |
| DTJ-17-5318 | Interspecific | Cherry | N57 | 2.7 | Double | 22 | Vein | 2* | 105 | 12 | N137 A | Lanceolate | 25 | |
| | hybrid | | B + C | | | | | | | | | | | |
| DTJ-17-5319 | Interspecific | Cherry | 86B | 2.6 | Double | 16 | Plain | 0* | 92 | 16 | N137 A | Lanceolate | 16 | |
| | hybrid | | | | | | | | | | | | | |
| DTJ-17-5320 | Interspecific | Cherry | 73A | 2.5 | Double | 12.6 | Vein | 9 | 112 | 3 | N137 B | Lanceolate | 20 | 1 |
| | hybrid | | | | | | | | | | | | | |
| DTJ-17-5321 | Interspecific | Cherry | N66 D | 2.3 | Single | 5 | Vein | 34 | 80 | 11 | N137 B | Lanceolate | 18 | 1 |
| | hybrid | | | | | | | | | | | | | |
| DTJ-17-5322 | Interspecific | Light | 69B/ | 2 | Double | 17 | Vein | 85 | 87 | 12 | N137 B | Lanceolate | 11.5 | 0.9 |
| | hybrid | Pink | N66 D | | | | | | | | | | | |
| DTJ-17-5323 | Interspecific | Cherry | 67C | 2.1 | Single | 5 | Vein | 80 | 76 | 16 | N137 B | Lanceolate | 22 | 0.7 |
| | hybrid | | | | | | | | | | | | | |
| DTJ-17-5324 | Interspecific | Cherry | 67B | 2.6 | Double | 29 | Plain | 23 | 77 | 18 | N137 A | Lanceolate | 18 | |
| | hybrid | | | | | | | | | | | | | |
| DTJ-17-5325 | Interspecific | Cherry | N66 B | 2 | Single | 5 | Plain | 70 | 90 | 7 | N137 B | Lanceolate | 16 | 1 |
| | hybrid | | | | | | | | | | | | | |
| DTJ-17-5326 | Interspecific | Dark | 64C | 2.1 | Single | 5 | Plain | 80 | 87 | 10 | 135 A | Lincar | 10.5 | |
| | hybrid | pink | | | | | | | | | | | | |
| DTJ-17-5327 | Interspecific | Cherry | 64C | 2.2 | Double | 20 | Vein | 13 | 60 | 15 | N137 A | Lanceolate | 13.5 | |
| | hybrid | | | | | | | | | | | | | |
| DTJ-17-5328 | Interspecific | Cherry | N66 C | 2.4 | Double | 20.6 | Plain | 23 | 86 | 7 | N137 B | Lincar | 21 | |
| | hybrid | | | | | | | | | | | | | |
| DTJ-17-5329 | Interspecific | Cherry | 68A | 2.1 | Double | 22.6 | Plain | 31 | 74 | 18 | N137 B | Lanceolate | 20 | |
| | hybrid | | | | | | | | | | | | | |
| DTJ-17-5330 | Interspecific | Light | 65C | 2.6 | Double | 22.3 | Plain | 7 | 81 | 12 | N137 C | Lanceolate | 20 | 0.5 |
| | hybrid | pink | | | | | | | | | | | | |
| DTJ-17-5331 | Interspecific | dark | 77B | 2.5 | Double | 10 | Plain | 6 | 30 | 3 | 139A | Lanceolate | 16 | 1.3 |
| | hybrid | pink | | | | | | | | | | | | |
| DTJ-17-5332 | Interspecific | Dark | N74 A | 2 | Double | 36 | Plain | 40 | 28 | 13 | N137 A | Lanceolate | 9 | 1.9 |
| | hybrid | pink | | | | | | | | | | | | |
| DTJ-17-5333 | Interspecific | Cherry | 64B | 2.5 | Double | 18 | Plain | 18 | 86 | 24 | N137 B | Lanceolate | 16 | 1 |
| | hybrid | | | | | | | | | | | | | |
| DTJ-17-5334 | Interspecific | Cherry | 67B | 3 | Double | 24.6 | Plain | 9 | 90 | 8 | N137 B | Lanceolate | 26 | 1 |
| | hybrid | | | | | | | | | | | | | |
| DTJ-18-6000 | Interspecific | Cherry | 64C | 2.5 | Double | 30 | Plain | 40 | 52 | 11 | N137 B | Lanceolate | 6 | 2 |
| | hybrid | | | | | | | | | | | | | |
| DTJ-18-6001 | Interspecific | Cherry | 64C | 2.1 | Single | 5 | Vein | 46 | 47 | 5 | N137 B | Lanceolate | 9.2 | |
| | hybrid | Cherry | 64C | 2.1 | Double | 27 | Plain | 44 | 58 | 6 | N137 B | Lanceolate | 9 | 1.5 |
| DTJ-18-6002 | Interspecific | | | | | | | | | | | | | |
| | hybrid | | | | | | | | | | | | | |

| | | Code-female | Genus-female | Code-male | Genus-female |
|---|---|---|---|---|---|
| Code | Genus | parental | parental | parental | parental |
| DTJ-17-5301 | Interspecific hybrid | DT-Z-175 | D. barbatus | | |
| DTJ-17-5302 | Interspecific hybrid | DT-Z-175 | D. barbatus | DTJ-16-4793 | D. japonicus |
| DTJ-17-5303 | Interspecific hybrid | DT-Z-171 | D. barbatus | DTJ-16-4787 | D. japonicus |
| | | | | | “Pink” |
| DTJ-17-5304 | Interspecific hybrid | DT-Z-173 | D. barbatus | DTJ-16-4794 | D. japonicus |
| DTJ-17-5305 | Interspecific hybrid | DT-Z-175 | D. barbatus | DTJ-16-4798 | D. japonicus |
| DTJ-17-5306 | Interspecific hybrid | DT-Z-171 | D. barbatus | DTJ-16-4787 | D. japonicus |
| | | | | | “Pink” |
| DTJ-17-5308 | Interspecific hybrid | DT-Z-173 | D. barbatus | DTJ-16-4795 | D. japonicus |
| DTJ-17-5309 | Interspecific hybrid | DT-Z-171 | D. barbatus | DTJ-16-4790 | D. japonicus |
| DTJ-17-5310 | Interspecific hybrid | DT-Z-171 | D. barbatus | DTJ-16-4796 | D. japonicus |
| DTJ-17-5311 | Interspecific hybrid | DT-Z-171 | D. barbatus | DTJ-16-4794 | D. japonicus |
| DTJ-17-5312 | Interspecific hybrid | DT-Z-171 | D. barbatus | DTJ-16-4794 | D. japonicus |
| DTJ-17-5314 | Interspecific hybrid | DT-Z-171 | D. barbatus | DTJ-16-4798 | D. japonicus |
| DTJ-17-5315 | Interspecific hybrid | DT-Z-122 | D. barbatus | | |
| DTJ-17-5316 | Interspecific hybrid | DT-Z-172 | D. barbatus | DTJ-16-4794 | D. japonicus |
| DTJ-17-5317 | Interspecific hybrid | DT-Z-175 | D. barbatus | DTJ-16-4800 | D. japonicus |
| DTJ-17-5318 | Interspecific hybrid | DT-Z-171 | D. barbatus | DTJ-16-4795 | D. japonicus |
| DTJ-17-5319 | Interspecific hybrid | DT-Z-175 | D. barbatus | DTJ-16-4793 | D. japonicus |
| DTJ-17-5320 | Interspecific hybrid | DT-Z-175 | D. barbatus | DTJ-16-4793 | D. japonicus |
| DTJ-17-5321 | Interspecific hybrid | DT-Z-172 | D. barbatus | | D. japonicus |
| DTJ-17-5322 | Interspecific hybrid | DT-Z-175 | D. barbatus | DTJ-16-4798 | D. japonicus |
| DTJ-17-5323 | Interspecific hybrid | DT-Z-171 | D. barbatus | DTJ-16-4798 | D. japonicus |
| DTJ-17-5324 | Interspecific hybrid | DT-Z-171 | D. barbatus | DTJ-16-4795 | D. japonicus |
| DTJ-17-5325 | Interspecific hybrid | DT-Z-171 | D. barbatus | DTJ-16-4787 | D. japonicus |
| | | | | | “Pink” |
| DTJ-17-5326 | Interspecific hybrid | DT-Z-173 | D. barbatus | DTJ-16-4789 | D. japonicus |
| DTJ-17-5327 | Interspecific hybrid | DT-Z-171 | D. barbatus | DTJ-16-4787 | D. japonicus |
| | | | | | “Pink” |
| DTJ-17-5328 | Interspecific hybrid | DT-Z-171 | D. barbatus | DTJ-16-4787 | D. japonicus |
| | | | | | “Pink” |
| DTJ-17-5329 | Interspecific hybrid | DT-Z-171 | D. barbatus | DTJ-16-4796 | D. japonicus |
| DTJ-17-5330 | Interspecific hybrid | DT-Z-175 | D. barbatus | DTJ-16-4988 | D. japonicus |
| DTJ-17-5331 | Interspecific hybrid | DT-Z-175 | D. barbatus | DTJ-16-4793 | D. japonicus |
| DTJ-17-5332 | Interspecific hybrid | DT-Z-171 | D. barbatus | DTJ-16-4796 | D. japonicus |
| DTJ-17-5333 | Interspecific hybrid | DT-Z-171 | D. barbatus | DTJ-16-4798 | D. japonicus |
| DTJ-17-5334 | Interspecific hybrid | DT-Z-175 | D. barbatus | DTJ-16-4792 | D. japonicus |

### Example 3

### Rooting Ability

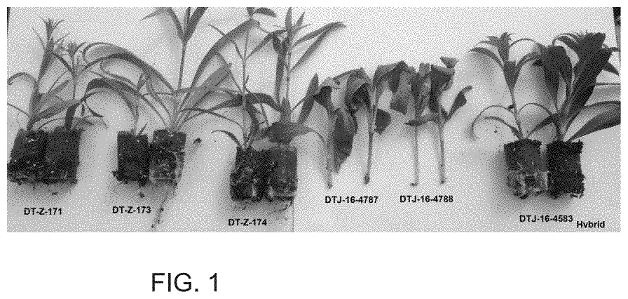

Due to its self-pollination character, D. japonicas was propagated and maintained from seeds. The rooting of cuttings from D. japonicas was hardly possible and it was well observed in a portion of the interspecific hybrids that their rooting ability is very poor and it was very difficult to sustain them vegetatively. Genetic variation in rooting ability was observed in hybrids population, thus selection for high rooting ability was performed on the new hybrids. The results are presented in Table 2 and FIG. 1.

TABLE 2
Genetic variation in rooting ability of the interspecific hybrids.

| | | | Number of | | % | % |
|---|---|---|---|---|---|---|
| | | Number of | rooted | Number of | Rooted | Flowering |
| Variety | Specie | cuttings | cuttings | Flowering | cuttings | cuttings |
| DT-Z-173 | D. barbatus | 26 | 25 | 15 | 96.2% | 57.7% |
| DT-Z-171 | D. barbatus | 9 | 6 | 0 | 66.7% | 0.0% |
| DTJ-17-5329 | Interspecific hybrid | 10 | 10 | 2 | 100.0% | 20.0% |
| DTJ-17-5313 | Interspecific hybrid | 15 | 13 | 0 | 86.7% | 0.0% |
| DTJ-17-5312 | Interspecific hybrid | 15 | 12 | 10 | 80.0% | 66.7% |
| DTJ-17-5331 | Interspecific hybrid | 10 | 8 | 3 | 80.0% | 30.0% |
| DTJ-16-4803 | Interspecific hybrid | 34 | 27 | 13 | 79.4% | 38.2% |
| DTJ-17-5319 | Interspecific hybrid | 14 | 10 | 6 | 71.4% | 42.9% |
| DTJ-17-5309 | Interspecific hybrid | 15 | 10 | 6 | 66.7% | 40.0% |
| DTJ-17-5310 | Interspecific hybrid | 15 | 10 | 10 | 66.7% | 66.7% |
| DTJ-17-5316 | Interspecific hybrid | 10 | 6 | 3 | 60.0% | 30.0% |
| DTJ-17-5318 | Interspecific hybrid | 15 | 9 | 3 | 60.0% | 20.0% |
| DTJ-17-5333 | Interspecific hybrid | 30 | 14 | 6 | 46.7% | 20.0% |
| DTJ-17-5330 | Interspecific hybrid | 11 | 5 | 9 | 45.5% | 81.8% |
| DTJ-16-4812 | Interspecific hybrid | 10 | 4 | 4 | 40.0% | 40.0% |
| DTJ-17-5317 | Interspecific hybrid | 28 | 10 | 2 | 35.7% | 7.1% |
| DTJ-17-5327 | Interspecific hybrid | 15 | 5 | 3 | 33.3% | 20.0% |
| DTJ-16-4808 | Interspecific hybrid | 10 | 3 | 0 | 30.0% | 0.0% |
| DTJ-17-5315 | Interspecific hybrid | 10 | 3 | 0 | 30.0% | 0.0% |

Rooting ability was presumably inherited from the D. barbatus species.

### Example 4

### Genotyping-by-Sequencing (GBS) Genetic Profiling and Genetic Diversity Analysis

A sample of 95 Dianthus varieties from 7 sub-species, including interspecific hybrids, were sampled for GBS (PstI/MspI dual digest) molecular analysis. 15795 SNP genetic markers were identified and anchored to the Dianthus caryophillis draft genome assembly. SNP calling Illumina reads were implemented on TASSEL software, after filtering under various quality control (QC) conditions a total of 1495 SNPs were selected with Minor Allelic Frequency (MAF)<5% and % missing genotypes across all samples <15%.

### Example 5

### Identification of Interspecific Hybrid Plants

In the GBS-based assay as described in Example 4 above, an optimized set of 4 SNP markers for species identification were achieved by detecting allelic variations in these types of markers. Joint use of these markers was shown to consistently distinguish the two parental species, D. barbatus and D. japonicas, and their hybrids. The SNPs markers named Sequence 1 (SEQ ID NO: 1) and Sequence 2 (SEQ ID NO: 2) are specific to D. japonicas and the SNPs markers named Sequence 3 (SEQ ID NO: 3) and Sequence 4 (SEQ ID NO: 4) are specific to D. barbatus.

TABLE 3
markers for identification of hybrids according to some embodiments of the
invention

| SNP Marker | SNP marker sequence |
|---|---|
| Sequence 1 (SEQ ID | TGCAGGTAATGGCTTTCGTGCTGCTGTTATTTCTTCCTCTTTTTTTGTGC(G\T)GAGCTTTAACTCG |
| NO: 1) | |
| Sequence 2 (SEQ ID | TGCAGA(C/T)AGCTAATTAATCAAATAAAGAGAGGATGAATGGCAGACAGATAATTAACAAATCGTA |
| NO: 2) | |
| Sequence 3 (SEQ ID | TGCAGGTCTACTACTTCCAAATGTATCTGGCTCTAGTACTCCTCGGGTTTTTACA(C\T)GGTCTTAT |
| NO: 3) | |
| Sequence 4 (SEQ ID | TGCAGAAACAAAAATTGGTTCCACAGGACTGCATTTAACATCTAAGA(C\T)CCTGTTGAAAATAAAT |
| NO: 4) | |

### Example 6

### Resistance to Fusarium oxysporum

One of the most severe problems affecting carnation (Dianthus caryophyllus L.), in most areas of the world where the crop is grown, is the soilborne pathogen fungus Fusarium oxysporum forma specialis dianthi (Fod) race 2 (Holley and Baker, 1991). The pathogen causes vascular wilt of carnation, a disease called Fusarium wilt. This disease causes extensive yield loss; symptoms consist of yellowing and wilting of branches that gradually progress until the plant dies, leading to deterioration in quality and quantity of marketable blooms and planting materials.

A new source of resistance to Fusarium oxysporum f.sp. dianthi, race 2, was discovered in D. japonicus and had been inherited to the interspecific hybrids. Artificial inoculation with the pathogen, as a bioassay, followed by phenotypic scoring was conducted in order to determine resistance. Results are presented in Table 4. Varieties of D. japonicas DTJ-16-4787 and DTJ-16-4788 that were used as male parents, were highly resistant to Fusarium oxysporum f.sp. dianthi, race 2, while the maternal varieties of D. barbatus, DT-Z-171, DT-Z-173 and DT-Z-174, displayed variable level of sensitivity. All the interspecific hybrids tested showed a very high level of resistance, presumably inherited from the parental specie of D. japonicas.

Materials and Methods

The monosporic isolates belonging to pathotype 2 of F. dianthi (Migheli et al., 1998) were cultivated on liquid casein hydrolysate at 24-26°, in agitation for 10-13 days. The cultures were then homogenized in order to diminish the most unrefined parts, and then diluted with deionized water until a concentration of fungal spores and of mycelium fragments of 1*10⁶ CFU/ml and 1*10⁴ CFU/ml was obtained. In these suspensions, a root dipping of the Dianthus vine cuttings was taking place in order to verify their susceptibility to different pathotypes of F. dianthi. The test made use of multiple cuttings for each line. . . . Subsequently the cuttings were transplanted in benches using a substrate disinfected with steam. The plants were grown in greenhouses made of glass and iron, in which, during the cultivation, the temperature was kept at no less than 18° C. and at no more than 35° C., tests were carried from April through June in Estazione Ed Assistenza Agricola, Regione Rolo 98, Abenga, Italy). During the cultivation period, the plants were subjected, when necessary, to periodic treatments with insecticides and acaricides; besides, some treatments with fungicides were also carried out, only through spraying the fungicides on the leaves and with active ingredients that are not effecting vìs-à-vìs F. dianthi. The surveys were carried out starting with the first symptoms of Fusarium wilt and were continued with a frequency period which varied from 7 to 15 days. In the course of each survey, the plants, which had died because of the pathogen attack, were counted and eliminated. During the last survey, a final evaluation was also carried out regarding the severity of the infection on the plants which were still alive, through the assignment of a sickness index from 0 (healthy plant) to 4 (dead plant).

TABLE 4
Bioassay results of varieties tested for resistance to Fusarium
oxysporum f.sp. dianthi, race 2.
Phenotypic scoring for disease index from 0 (healthy plant) to 4
(dead plant).

| | | Fungal | | Fungal | |
|---|---|---|---|---|---|
| | | Concentration of 1*10(4)CFU/ml | | Concentration of 1*10(6)CFU/ml | |
| | | | Average | | Average |
| | | % | disease | % | disease |
| | | surviving | index/ | surviving | index/ |
| Variety | Specie | plants | plant | plants | plant |
| DTP-Z-171 | D. barbatus | 40 | 2.4 | 0 | 4 |
| DTP-Z-173 | D. barbatus | 33.3 | 2.7 | 33.3 | 2.7 |
| DTP-Z-174 | D. barbatus | 75.0 | 1.0 | 0.0 | 4.0 |
| DTJ-16-4787 | D. japonicas | 100.0 | 0.0 | 100.0 | 0.0 |
| DTJ-16-4788 | D. japonicas | 100.0 | 0.0 | 100.0 | 0.0 |
| DTJ-16-4808 | Interspecific | 100.0 | 0.0 | 100.0 | 0.0 |
| | hybrid | | | | |
| DTJ-17-5308 | Interspecific | 100.0 | 0.0 | 100.0 | 0.0 |
| | hybrid | | | | |
| DTJ-17-5317 | Interspecific | 100.0 | 0.0 | 100.0 | 0.0 |
| | hybrid | | | | |
| DTJ-17-5325 | Interspecific | 100.0 | 0.0 | 100.0 | 0.0 |
| | hybrid | | | | |
| DTJ-17-5329 | Interspecific | 100.0 | 0.0 | 100.0 | 0.0 |
| | hybrid | | | | |
| DTJ-17-5330 | Interspecific | 100.0 | 0.0 | 100.0 | 0.0 |
| | hybrid | | | | |
| DTJ-17-5334 | Interspecific | 100.0 | 0.0 | 100.0 | 0.2 |
| | hybrid | | | | |

### Example 7

### Heat Tolerance

Heat tolerance is an important goal in breeding Dianthus, since varieties which will continue flowering with quality stems throughout the summer season, will enable the growers to extend their season and get more yield per square meter. D. japonicus possesses unusually high tolerance to high temperature: whereas, D. barbatus begins to show significant heat stress at 30° C., D. japonicus only shows mild symptoms at temperatures nearing 40° C. The interspecific hybrids selected according to some embodiments of the invention display a phenotypic appearance like their parental specie of D. japonicas with regard to heat tolerance.

### Example 8

### Induction of Ploidy During the Breeding Process

Methods:

Chemical/Physical Induced Polyploidy

I. Anti-mitotic substance: Colchicine

Dosage: colchicine 20-300 μM
Incubation time: 10-120 h
Medium: solid/liquid MS
Explant type: in-vitro or in-vivo
Plant material: Stems with axillary bud, leaf segments, seeds

II. Anti-mitotic substance: Oryzalin

Dosage: oryzalin 5-150 μM
Incubation time: 10-120 h
Medium: solid/liquid MS
Explant type: invitro or invivo
Plant material: Stems with axillary bud, leaf segments, seeds

III. chemical mutagens to induce polyploid mutation: an alkylating agent, hydroxylamine NH₂OH, base analogues, agents that form DNA adducts, DNA intercalating agents, DNA crosslinkers, oxidative damage inducers and nitrous acids.

IV. Physical induced polyploidy: Stems with axillary bud, leaf segments and seeds are exposed to different dosages of radiations (fast neutron, x-ray, gamma radiation).

Plant Material

The source of the plant material that was subjected to induction treatment are: D. japonicus, D. barbatus and the D. japonicus X D. barbatus hybrids. See detailed varieties in Table 5 below.

TABLE 5

| Code | Genus | Code | Genus |
|---|---|---|---|
| DTJ-17-5301 | Interspecific hybrid | DTJ-16-4793 | D. japonicus |
| DTJ-17-5302 | Interspecific hybrid | DTJ-16-4787 | D. japonicus |
| | | | “Pink” |
| DTJ-17-5303 | Interspecific hybrid | DTJ-16-4794 | D. japonicus |
| DTJ-17-5304 | Interspecific hybrid | DTJ-16-4798 | D. japonicus |
| DTJ-17-5305 | Interspecific hybrid | DTJ-16-4795 | D. japonicus |
| DTJ-17-5306 | Interspecific hybrid | DTJ-16-4790 | D. japonicus |
| DTJ-17-5308 | Interspecific hybrid | DTJ-16-4796 | D. japonicus |
| DTJ-17-5309 | Interspecific hybrid | DTJ-16-4800 | D. japonicus |
| DTJ-17-5310 | Interspecific hybrid | DTJ-16-4789 | D. japonicus |
| DTJ-17-5311 | Interspecific hybrid | DTJ-16-4988 | D. japonicus |
| DTJ-17-5312 | Interspecific hybrid | DTJ-16-4792 | D. japonicus |
| DTJ-17-5314 | Interspecific hybrid | | |
| DTJ-17-5315 | Interspecific hybrid | DT-Z-175 | D. barbatus |
| DTJ-17-5316 | Interspecific hybrid | DT-Z-171 | D. barbatus |
| DTJ-17-5317 | Interspecific hybrid | DT-Z-173 | D. barbatus |
| DTJ-17-5318 | Interspecific hybrid | DT-Z-122 | D. barbatus |
| DTJ-17-5319 | Interspecific hybrid | DT-Z-172 | D. barbatus |

| | |
|---|---|
| DTJ-17-5320 | Interspecific hybrid |
| DTJ-17-5321 | Interspecific hybrid |
| DTJ-17-5322 | Interspecific hybrid |
| DTJ-17-5323 | Interspecific hybrid |
| DTJ-17-5324 | Interspecific hybrid |
| DTJ-17-5325 | Interspecific hybrid |
| DTJ-17-5326 | Interspecific hybrid |
| DTJ-17-5327 | Interspecific hybrid |
| DTJ-17-5328 | Interspecific hybrid |
| DTJ-17-5329 | Interspecific hybrid |

| | |
|---|---|
| DTJ-17-5330 | Interspecific hybrid |
| DTJ-17-5331 | Interspecific hybrid |
| DTJ-17-5332 | Interspecific hybrid |
| DTJ-17-5333 | Interspecific hybrid |
| DTJ-17-5334 | Interspecific hybrid |

The plant material that was subjected to treatment was grown and closely analyzed. Pollen from the treated plants were collected and applied on other treated plants and on non-treated plants.

Progeny of all treated plants was examined as well.

Phenotypic selection is the first step to identify the polyploid varieties. Plants presenting an abnormal phenotype are closely examined.

Plants presenting low vigor and/or low vitality are suspected of suffering of unwanted mutation, primarily deletion of essential genes or chromosomes.

Plants presenting higher vigor, larger organs, different plant structure, smaller organs, unusual color or color pattern, change in fragrance, different flower structure compared to the parents etc. are further examined and samples of their tissue are collected and sent to Flow Chemistry Evaluation to assess the level of ploidy.

Some plants present changes in phenotype at the level of the whole plant, other present the change in phenotype in parts of the plants (also known as chimera—e.g. only one stem or a branch show the altered phenotype).

Some plants show only temporal change in phenotype. In others the phenotype is stable throughout generations (e.g., more than 1 or 2 generations).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

1. Jürgens. A Witt. T Gottsberger. G (2003) Flower scent composition in Dianthus and Saponaria species (Caryophyllaceae) and its relevance for pollination biology and taxonomy. Biochemical systematics and ecology Vol. 31 Issue. 4 p. 345-357.
2. M. Nimura J. Kato M. Mii K. Moriok (2003) Unilateral compatibility and genotypic difference in crossability in interspecific hybridization between Dianthus caryophyllus L. and Dianthus japonicus Thunb. Theor Appl Genet (2003) 106:1164-1170.
3. Kanda M (1992) Ovule culture for hybridization between carnation and the genus Diantus. J JPN Soc hortic Sci 61:464-465.
4. Onozaki, T & Ikeda, H & Yamaguchi, T & Himeno, M. (1998). Introduction of bacterial wilt (Pseudomonas caryophylli) resistance in dianthus wild species to carnation. Acta Horticulturae. 454. 127-132. 10.17660/ActaHortic.1998.454.15.
5. Nakano M, Mii M. (1993) Somatic hybridization between Dianthus chinesis and D. barbatus through protoplast fusion. Theor Appl Genet 86:1-5.
6. Tukamoto Y (1968) Dianthus. Encyclopedia of Horticulture, vol. 2 (in Japanese). Seibundoshinkosya, Ltd.
7. Kagito N, Tuchiya Y (1968) Outbreak and protection of carnation bacterial wilt (in Japanese). Plant Protect 22:67-70.
8. Holley D W, Baker R. (1991) Carnation production II. Kendall Hunt publishing company Dubuque 156pp.
9. Q. Migheli, E. Briatore, and A. Garibaldi (1998) Use of random amplified polymorphic DNA (RAPD) to identify races 1, 2, 4 and 8 of Fusarium oxysporum f. sp. dianthi in Italy. European Journal of Plant Pathology, vol. 104, no. 1, pp. 49-57.
10. M. Mii (2009) Breeding of Ornamental Plants through Interspecific Hybridization Using Advanced Techniques with a Special Focus on Dianthus, Primula, Cosmos and Kalanchoe Proc. 23rd Intl. Eucarpia Symp. (Sec. Ornamentals) on “Colourful Breeding and Genetics” Eds.: J. M. van Tuyl and D. P. de Vries Acta Hort. 836, ISHS 2009.

1. An interspecific Dianthus plant comprising a genomic sequence distinctive of D. japonicus and a genomic sequence distinctive of Dianthus species not being D. japonicus, said plant exhibiting resistance to Fusarium oxysporum f.sp. dianthi, race 2 which is higher than that of said Dianthus species.

2. An interspecific Dianthus plant comprising a genomic sequence distinctive of D. barbatus and a genomic sequence distinctive of D. japonicus.

3. The interspecific plant of claim 2, wherein flowers of the interspecific plant are endowed with a sweet scent and/or higher shelf life compared to that of a D. barbatus parent or about the same as that of a D. japonicus parent of the interspecific plant.

4. The interspecific plant of claim 2, exhibiting:
resistance to Fusarium oxysporum f.sp. dianthi, race 2 which is higher than that of a D. barbatus parent or at least about the same as that of a D. japonicus parent of the interspecific plant;
heat tolerance which is higher than that of a D. barbatus parent of the interspecific plant or about the same or higher than that of a D. japonicus parent;
flower size which is larger than that of a D. japonicus parent of the interspecific plant;
flower size of at least 1 cm;
flower size of 1-6 cm;
stem length exceeding 60 cm;
stem length of 60-120 cm;
a stem length of 15-60 cm;
single flower type;
double flower type;
semi-double flower type;
flower color selected from the group consisting of dark pink, cerise, light pink, purple and cream white;
flower pattern selected from the group consisting of plain, vein and bicolor;
flower type selected from the group consisting of a head type flower and a spray type flower;
green flower-like structure; and/or
rooting ability at least as good as that of a D. barbatus parent of the interspecific plant.

5-19. (canceled)

20. The interspecific plant of claim 2, being sterile or fertile.

21. (canceled)

22. The interspecific plant of claim 1, wherein said genomic sequence distinctive of D. barbatus is as set forth in Sequence 3 (SEQ ID NO: 3) or Sequence 4 (SEQ ID NO: 4).

23. The interspecific plant of claim 1, wherein said genomic sequence distinctive of D. japonicus is as set forth in Sequence 1 (SEQ ID NO: 1) or Sequence 2 (SEQ ID NO: 2).

24. The interspecific plant of claim 2, being polyploid.

25. A cut flower of the plant of claim 2.

26. A cutting of the plant of claim 2.

27. A hybrid seed forming the plant of claim 2.

28. A tissue culture of the plant of claim 2.

29. A potted flower of the plant of claim 2.

30. A method of producing the interspecific Dianthus plant of claim 2, the method comprising:
(a) selecting from a plurality of D. japonicus plants an early flowering D. japonicus plant;
(b) crossing said early flowering D. japonicus plant with a Dianthus species not being D. japonicus so as to obtain the interspecific Dianthus plant.

31. A method of producing the interspecific Dianthus plant of claim 2, the method comprising:
(a) selecting from a plurality of D. japonicus plants an early flowering D. japonicus plant;
(b) crossing said early flowering D. japonicus plant with a D. barbatus plant so as to obtain the interspecific Dianthus plant.

32-37. (canceled)

38. A method of growing the interspecific Dianthus plant of claim 2, the method comprising, sowing the seed of the interspecific Dianthus plant or planting the cutting of the interspecific Dianthus plant under conditions which allow growth of the interspecific Dianthus plant.

39. A D. japonicus plant or a part thereof, representative seeds of said plant having been deposited under the Budapest Treaty at the NCIMB under NCIMB 43450 (DTJ-16-4748) on Aug. 2, 2019, progeny or hybrids of the plant.

40. The interspecific plant of claim 2, wherein said genomic sequence distinctive of D. barbatus is as set forth in Sequence 3 (SEQ ID NO: 3) or Sequence 4 (SEQ ID NO: 4).

41. The interspecific plant of claim 2, wherein said genomic sequence distinctive of D. japonicus is as set forth in Sequence 1 (SEQ ID NO: 1) or Sequence 2 (SEQ ID NO: 2).


SEQUENCE LISTING (4 sequences)

SEQ ID NO: 1 - DNA, 64 bp, Artificial Sequence
  SNP marker sequence
  misc_feature (51)..(51): n is g or t
tgcaggtaatggctttcgtgctgctgttatttcttcctctttttttgtgcngagctttaactcg

SEQ ID NO: 2 - DNA, 64 bp, Artificial Sequence
  SNP marker sequence
  misc_feature (7)..(7): n is c or t
tgcaganagctaattaatcaaataaagagaggatgaatggcagacagataattaacaaatcgta

SEQ ID NO: 3 - DNA, 64 bp, Artificial Sequence
  SNP marker sequence
  misc_feature (56)..(56): n is c or t
tgcaggtctactacttccaaatgtatctggctctagtactcctcgggtttttacanggtcttat

SEQ ID NO: 4 - DNA, 64 bp, Artificial Sequence
  SNP marker sequence
  misc_feature (48)..(48): n is c or t
tgcagaaacaaaaattggttccacaggactgcatttaacatctaagancctgttgaaaataaat